(19) United States
(12) Patent Application Publication
(10) Pub. No.: US 2010/0010366 A1
(43) Pub. Date: Jan. 14, 2010

(54) METHOD TO EVALUATE PSYCHOLOGICAL RESPONSES TO VISUAL OBJECTS

(76) Inventor: Richard Bernard Silberstein, Victoria (AU)

Correspondence Address: MASTERMIND IP LAW PC, 421-A SANTA MARINA COURT, ESCONDIDO, CA, 92029, US

(21) Appl. No.: 12/520,860

(22) PCT Filed: Dec. 22, 2006
(86) PCT No.: PCT/AU2006/002005

§ 371 Date: Jun. 22, 2009

(51) Int. Cl. A61B 5/0484; A61B 5/00

(52) U.S. Cl. 600/544; 600/558

(57) ABSTRACT

A method of evaluating the response of a subject to visual features of a visual display, the method including the steps of: (a) presenting a visual display having particular visual features to the subject during a first period; (b) determining brain activity of the subject during the first period; (c) presenting reference display material to a subject during a second period; (d) determining reference brain activity of the subject during the second period; (e) tracking the gaze position of at least one of the eyes of the subject on the visual display during the first period; and (f) evaluating the response of the subject to particular visual features of the visual display by determining differences in brain activity determined between steps (b) and (d) when the gaze of the subject is directed at the particular features.

There is a commercial imperative to enhance the effectiveness of various types of visual displays, web sites, print advertising as well as enhancing the attractiveness of product design and packaging. At present, eye movement technology is one of the methods used to evaluate individuals' psychological response to text layout, print advertising, product design and web site layout. While eye movement technology gives an indication of where gaze or visual attention is directed, it gives no indication of the psychological response associated with the direction of the gaze.

This invention discloses a method that combines brain activity and eye position to indicate the psychological response associated with visual attention to specific components of the visual image or product. This would enable advertisers, manufacturers, web site developers and architects the opportunity modify and hence improve the visual material such as text, billboard, product, building or web site. These will be collectively termed ‘visual objects’ in the description which follows.

Brain activity and gaze position are simultaneously measured while subjects view any type of visual display such as a page of text, an advertising billboard, object, a product such as a car or a perfume bottle or a building or a part of a building. The image may comprise an object, a display on a video monitor or a ‘virtual reality’ display. The term “visual display” is intended to encompass all of the foregoing.

To determine the brain activity associated with a specific visual image and gaze location, individual subject brain activity is averaged for all points in time when the gaze position is in the vicinity of a specific part of the image. For each subject, this will yield a set of mean brain activity measures associated with a specific part of the image. Brain activity for a given part of the image is then averaged across all the subjects or subset of subjects. The likely effectiveness of the image or product depends on the extent that the image elicits the desired emotional or cognitive state.

More specifically the invention provides a method of evaluating the response of a subject to visual features of a visual display, the method including the steps of:

(a) presenting a visual display having particular visual features to the subject during a first period;

(b) determining brain activity of the subject during the first period;

(c) presenting reference display material to a subject during a second period;

(d) determining reference brain activity of the subject during the second period;

(e) tracking the gaze position of at least one of the eyes of the subject on the visual display during the first period; and

(f) evaluating the response of the subject to particular visual features of the visual display by determining differences in brain activity determined between steps (b) and (d) when the gaze of the subject is directed at the particular features.

The invention also provides a system for evaluating the response of a subject to visual features of a visual display, the system including:

(a) display means for displaying said visual features to the subject;

(b) means for determining brain activity of the subject at predetermined scalp sites of the subject;

(c) gaze tracking means for determining the gaze position of the subject on said display means;

(d) detecting means for detecting when the gaze position of the subject impinges on selected visual features; and

(e) averaging means for calculating average values of brain activity for each of the selected visual features when the detecting means detects that the gaze position of the subject impinges on the respective selected visual features.

In the event that the image constitutes a billboard or print advertisement, the key psychological measures are the levels of attention, the strength of the emotional response and the extent to which the key messages are encoded in long-term memory.

If the image constitutes an object or product such as an item of furniture, a car or a view of a room, the key psychological measures may be engagement, attention, desirability, emotional intensity and attraction.

### Engagement

The extent to which an image or a component of an image engages the subject is given by is given by the weighted mean brain activity at prefrontal sites while subjects are viewing the image or the part of the image. The brain activity measures that indicate the level of engagement are given by the following expression.

Engagement=(b₁*brain activity at electrode F₃+b₂*brain activity at electrode Pₚ₁+b₃*brain activity advance at electrode F₄+b₄*brain activity at electrode Fₚ₂) Equation 1

where b₁=0.1, b₂=0.4, b₃=0.1, b₄=0.4

If inverse mapping techniques are used, the relevant expression is:

Engagement=(d₁*brain activity at right orbito frontal cortex (in vicinity of Brodman area 11)+d₂*brain activity at right dorso-lateral prefrontal cortex (in vicinity of Brodman area 9)+d₃*brain activity at left orbito frontal cortex (in vicinity of Brodman area 11)+d₄*brain activity at left dorso-lateral prefrontal cortex (in vicinity of Brodman area 9)) Equation 2

where: d₁=0.1, d₂=0.4, d₃=0.1, d₄=0.4

Other psychological measures and their brain activity indicators that are of relevance include:

### Visual Attention

Visual attention associated with an image or part of an image is indicated by increased brain activity at left and right occipital recording sites. In the International 10-20 system that labels recording sites on the brain, the positions referred to above correspond to the vicinity of O₁ and O₂. If activity in deeper parts of the brain are assessed using inverse mapping techniques such as BESA, EMSE or LORETA in combination with either electrical or magnetic recordings or SSVEP or SSVER, the relevant location in the left cerebral cortex is the vicinity of the left and right occipital lobe.

### Desirability

In particular, the desirability associated with the image or part of an image of a product is indicated by increased brain activity at left and right parietal recording sites during the initial period. In the International 10-20 system that labels recording sites on the brain, the positions referred to above correspond to the vicinity of P₃ and P₄. If activity in deeper parts of the brain are assessed using inverse mapping techniques such as BESA, EMSE or LORETA in combination with either electrical or magnetic recordings or SSVEP or SSVER, the relevant location in the left cerebral cortex is the vicinity of the right intraparietal area.

### Emotional Intensity

The emotional intensity associated with an Image or product or a component of an image or product is indicated by increased brain activity at right parieto-temporal region, preferable approximately equidistant from right hemisphere electrodes O₂, P₄ and T₆ during the initial period. If inverse mapping techniques are used, the relevant location in the right cerebral cortex is the vicinity of the right parieto-temporal junction.

### Long Term Memory

How well various parts of the text or images are stored or encoded in long-term memory is indicated by increased brain activity at left and right temporal sites in the vicinity of T₅ and T₆ and also at right frontal sites equidistant between C₄, F₄ and F₈ and also at left frontal sites equidistant between C₃, F₃ and F₇ during the initial period. If inverse mapping techniques are used, the relevant locations in the left and right temporal lobes in the vicinity of Brodman's area 20 and in the left and right frontal cortex in the vicinity of Brodmans areas 6, 44, 45, 46 and 47.

### Attraction/Repulsion

The extent to which individuals are attracted or repelled by the various parts of the product image is given by the difference between brain activity at left frontal/prefrontal and right frontal/prefrontal regions. Attraction is indicated by a larger activity in the left hemisphere compared to the right while repulsion is indicated by greater activity in the right hemisphere compared to the left.

Attraction=(a₁*brain activity recorded at electrode F₃+a₂*brain activity recorded at electrode Fₚ₁−a₃*brain activity recorded at electrode F₄−a₄*brain activity recorded at electrode Fₚ₂) Equation 3

where a₁=a₂=a₃=a₄=1.0

A positive value for the attraction measure is associated with the participants finding the image or product attractive and liked while a negative measure is associated with repulsion or dislike.

If inverse mapping techniques are used, the relevant expression is:

Attraction=(c₁*brain activity at right orbito-brain activity at frontal cortex (in vicinity of Brodman area 11)+c₂*brain activity at right dorso-lateral prefrontal cortex (in vicinity of Brodman area 9)+c₃*brain activity at left orbito frontal cortex (in vicinity of Brodman area 11)+c₄*brain activity at left dorso-lateral prefrontal cortex (vicinity of Brodman area 9)) Equation 4

where c₁=1, c₂=1, c₃=1, c₄=1

### Measuring Gaze Position

A number of techniques whose principles are in the public domain are available to measure gaze position. The most suitable for use in the method of the invention utilizes a commercially available system such as ‘TrackIR’ produced by Natural Point Inc, of Corvallis, Oreg. 97339, USA. This comprises an infra-red camera mounted on a helmet worn by the subject. The infra-red camera coupled with an infra-red landmarks near the visual display enable head position to be determined. Eye position within the orbit of the eye can be measured by infra red oculography (Reutens et al. 1988A and 1988B Stimulation and Recording of Dynamic Pupillary Reflex: the IRIS Technique Part 1 and Part 2 Medical and Biological Engineering and Computing, 26: 20-32). Commercial systems to measure eye position such as the Skalar Iris Limbus Tracker are available from Cambridge Research Systems Ltd., 80 Riverside Estate, Sir Thomas Longley Road, Rochester, Kent ME2 4BH England. Infra-red oculography lends itself best to the use of the steady state visually evoked potential (SSVEP) as the infra-red light emitting diodes and photo-transistors can be incorporated into the SSVEP visor. Combining head position information derived from the camera with eye position from the infra-red oculography enables one to determine gaze position. Using infra-red oculography system in combination with the TrackIR head position system enables gaze position to be determined to an accuracy of 0.25 degrees and updated every 40 msec.

### Measuring Brain Activity

A number of methods are available for measuring brain activity. The main feature they must possess is adequate temporal resolution or the capacity to track the rapid changes in brain activity. Spontaneous brain electrical activity or the electroencephalogram (EEG) or the brain electrical activity evoked by a continuous visual flicker that is the Steady State Visually Evoked (SSVEP) are two examples of brain electrical activity that can be used to measure changes in brain activity with sufficient temporal resolution. The equivalent spontaneous magnetic brain activity or the magnetoencephalogram (MEG) and the brain magnetic activity evoked by a continuous visual flicker Steady State Visually Evoked Response (SSVER).

### Electroencephalogram and Magnetoencephalogram (EEG and MEG)

The EEG and MEG are the record of spontaneous brain electrical and magnetic activity recorded at or near the scalp surface. Brain activity can be assessed from the following EEG or MEG components.

### 1. Gamma or High Frequency EEG or MEG Activity

This is generally defined as EEG or MEG activity comprising frequencies between 35 Hz and 80 Hz. Increased levels of Gamma activity are associated with increased levels of brain activity, especially concerned with perception (Fitzgibbon S P, Pope K J, Mackenzie L, Clark C R, Willoughby J O. Cognitive tasks augment gamma EEG power Clin Neurophysiol. 2004: 115:1802-1809).

If scalp EEG gamma activity is used as the indicator of brain activity, the relevant scalp recording sites are listed above. If EEG gamma activity at the specific brain regions listed above is used as the indicator brain activity then inverse mapping techniques such as LORETA are preferably used (Pascual-Marqui R, Michel C, Lehmann D (1994): Low Resolution Electromagnetic Tomography: A New Method for Localizing Electrical Activity in the Brain Int J Psychophysiol 18:49-65).

If MEG gamma activity at the specific brain regions listed above is used as the indicator of brain activity, then the multi-detector MEG recording system must be used in conjunction with an MEG inverse mapping technique (see Uutela K, Ha\ddot{}ma\ddot{}la\ddot{}inen M, Somersalo E (1999): Visualization of Magnetoencephalographic Data Using Minimum Current Estimates, Neuroimage 10.173-180 and Fuchs M, Wagner M, Kohler T, Wischmann H A (1999): Linear and Nonlinear Current Density Reconstructions. J Clin Neurophysiol 16:267-295).

### 2. Frequency of EEG or MEG Alpha Activity

Brain activity may also be indexed by changes in the frequency of the ongoing EEG or MEG in the alpha frequency range (8.0 Hz-13.0 Hz). Increased frequency is an indication of increased activity. The frequency needs to me estimated with high temporal resolution. Two techniques that can be used to measure ‘instantaneous frequency’ are complex demodulation (Walter D, The method of Complex Demodulation Electroencephalog. Clin. Neurophysiol. 1968: Suppl 27:53-7) and the use of the Hilbert Transform (Leon Cohen, “Time-frequency analysis”, Prentice-Hall, 1995). Increased brain activity is indicated by an increase in the instantaneous frequency of the EEG in the alpha frequency range.

If the frequency of scalp EEG alpha activity is used as the indicator of brain activity, the relevant scalp recording sites are listed above. If the frequency of EEG alpha activity at the specific brain regions listed above is used as the indicator brain activity then inverse mapping techniques such as LORETA are preferably used (Pascual-Marqui R, Michel C, Lehmann D (1994): Low Resolution Electromagnetic Tomography: A New Method for Localizing Electrical Activity in the Brain Int J Psychophysiol 18:49-65).

If the frequency of MEG alpha activity at the specific brain regions listed above is used as the indicator of brain activity, then the multi-detector MEG recording system must be used in conjunction with an MEG inverse mapping technique (see Uutela K, Ha\ddot{}ma\ddot{}la\ddot{}inen M, Somersalo E (1999): Visualization of Magnetoencephalographic Data Using Minimum Current Estimates, Neuroimage 10:173-180 and Fuchs M, Wagner M, Kohler T, Wischmann H A (1999): Linear and Nonlinear Current Density Reconstructions. J Clin Neurophysiol 16:267-295).

### 3. SSVEP or SSVER Phase as an Indicator of Brain Activity

Brain activity may also be indicated by the phase of the Steady State Visually Evoked Potential (SSVEP) or the Steady State Visually Evoked Response (SSVER).

U.S. Pat. Nos. 4,955,938, 5,331,969 and 6,792,304 (the contents of which are hereby incorporated herein by reference) disclose technique for obtaining a steady state visually evoked potential (SSVEP) from a subject. This technique can also be used to obtain a steady state visually evoked response (SSVER). These patents disclose the use of Fourier analysis in order to rapidly obtain the SSVEP and SSVER phase and changes thereto.

The invention will now be further described with reference to the accompanying drawings, in which:

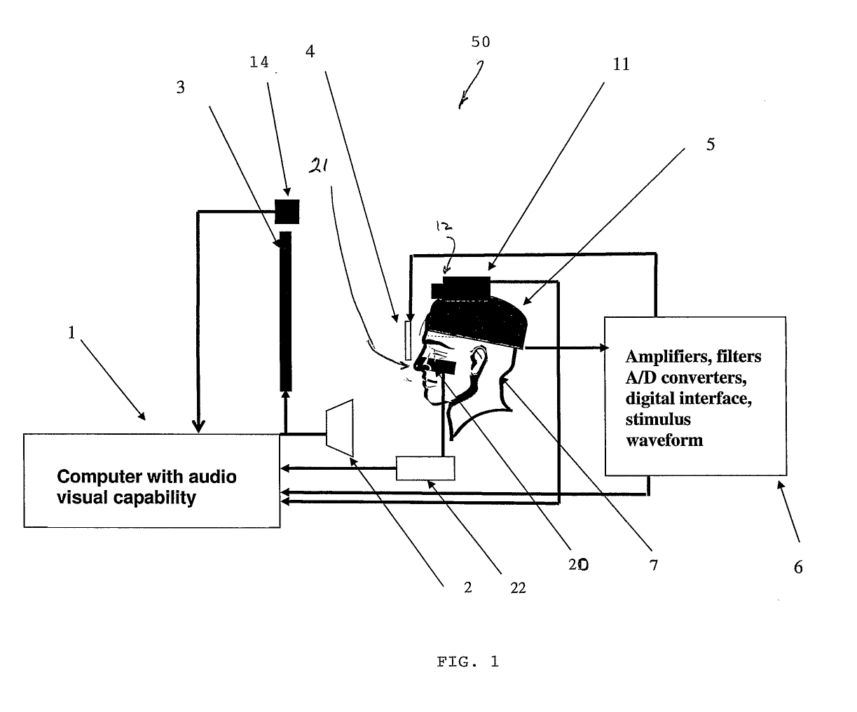

FIG. 1 is a schematic diagram of a system of the invention;

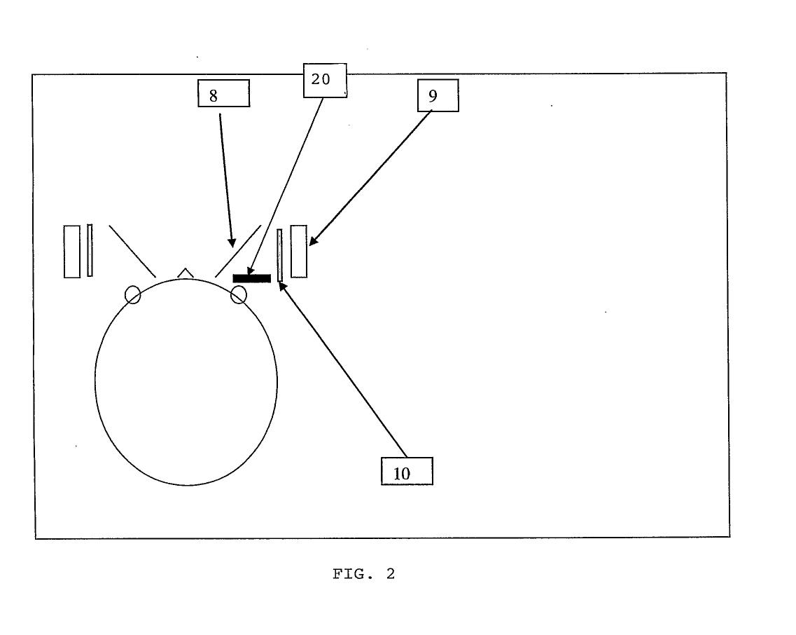

FIG. 2 is a schematic view showing in more detail the manner in which visual flicker stimuli are presented to a subject including the location of the infra-red diode and infra-red transistor;

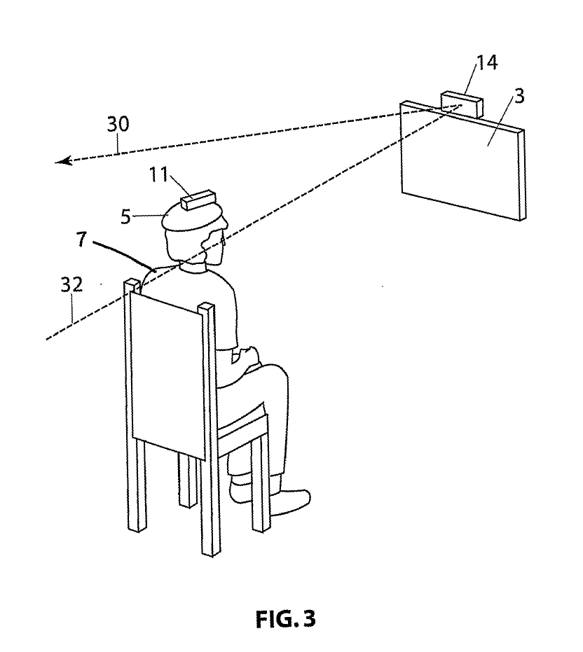

FIG. 3 is a schematic view of the operation of the TrackIR head tracking system;

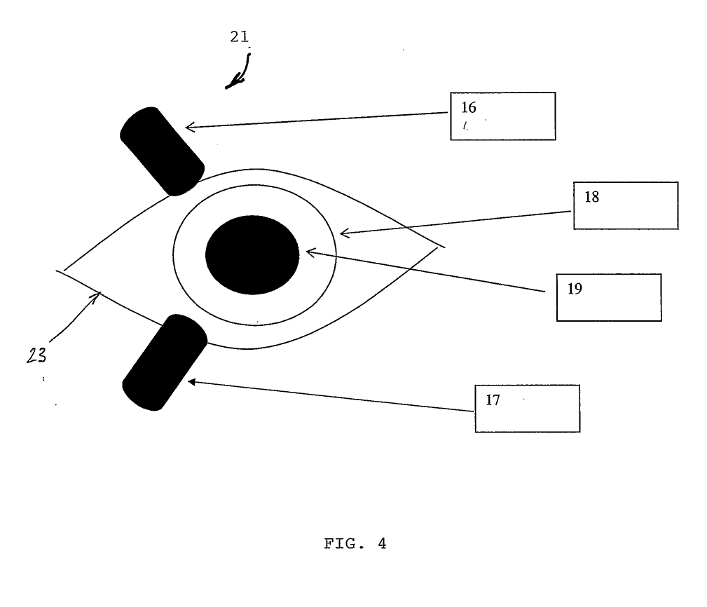

FIG. 4 is a schematic view of the locations of the infra-red diode and infra-red transistor for the infra-red oculography system;

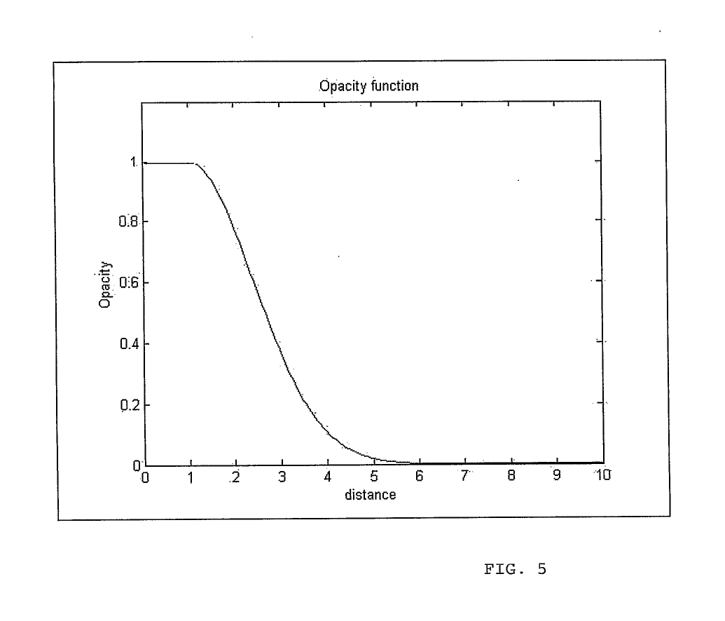

FIG. 5 is a diagrammatic representation showing opacity as a function of radius of a screen which is used in the system of the invention;

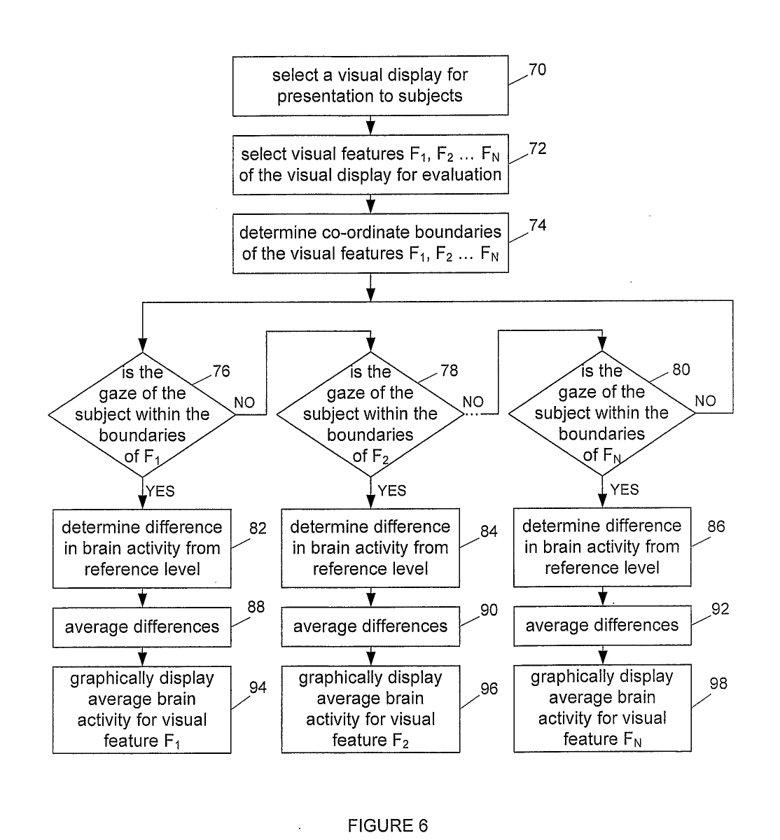

FIG. 6 is a flowchart illustrating a typical way in which features of a visual display are evaluated, in accordance with the method of the invention;

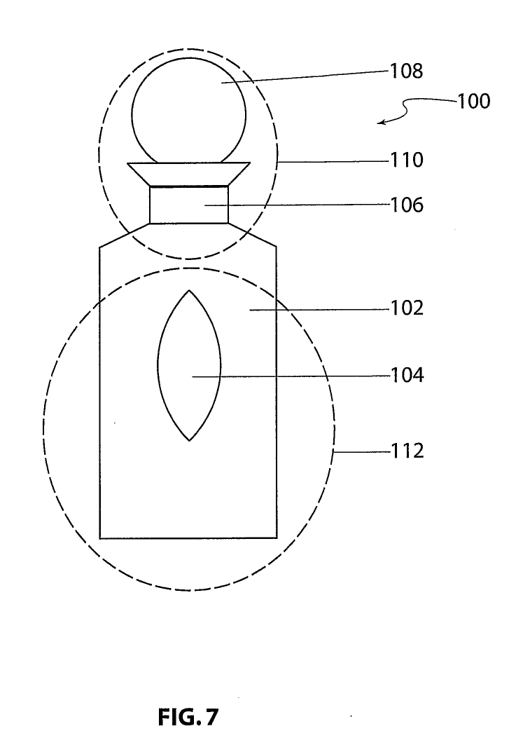

FIG. 7 shows an example of a visual object in the form of a perfume bottle; and

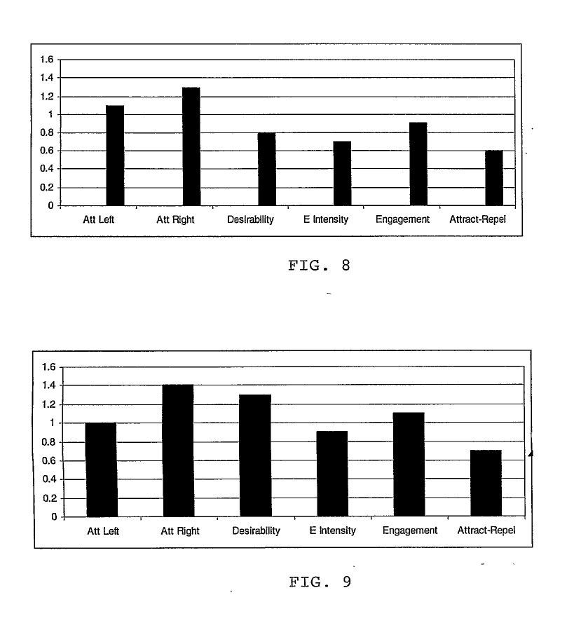

FIGS. 8 and 9 are graphs which show levels of psychological responses to parts of a visual object.

FIG. 1 schematically illustrates a system 50 for determining the response of a subject or player to a computer game presented on a video screen 3 and loudspeaker 2. The system includes a computer 1 which controls various parts of the hardware and also performs computation on signals derived from the brain activity of the subject 7, as will be described below. The computer 1 also presents the computer game which can be presented to the subject 7 on the screen 3 and/or through the loudspeaker 2.

The subject 7 to be tested are fitted with a headset 5 which includes a plurality of electrodes for obtaining brain electrical activity from various sights on the scalp of the subject 7. The system includes a head tracking system 12 which preferably is the TrackIR head position tracking system referred to above and includes a head mounted camera 11, cables connecting the camera 11 to the computer 1 and software running on the computer 1.

FIG. 3 schematically illustrates the operation of the head tracking system 12 in more detail. The system includes an infra-red light reference source 14 which produces at least two beams 30 and 32 of infra-red radiation. The beams are oriented at predetermined directions relative to one another and are generally directed at the subject 7. The head mounted camera 11 receives components of the two beams depending on the orientation of the head of the subject and from this information, the supplied software can compute the position of the head relative to the screen 3. The output from the camera 11 is coupled to the computer 1 and the software is arranged to sample the video output from the camera 11 at a predetermined sampling rate, say 20 times per second in order to provide adequate temporal resolution of the position of the subject's head relative to the screen 3.

In the event that the SSVER is used, the recording electrodes in the headset 5 are not used and a commercial MEG recording system such as the CTF MEG System manufactured by VSM MedTech Ltd of 9 Burbidge Street Coquitlam, BC, Canada, can be used instead. The headset includes a visor 4 which includes half silvered mirrors 8 and white light Light Emitting Diode (LED) arrays 9, as shown in FIG. 2.

The half silvered mirrors are arranged to direct light from the LED arrays 9 towards the eyes of the subject 7.

The system 50 also includes an oculography or eye tracking system 21 which is used to track the position of the subject's left or right eye so that this information combined with the output from the head position tracking system can be used to accurately determine the position of the gaze of the subject 7 relative to the centre of the screen 3. The eye tracking system 21 may be the scalar Iris Limbus Tracker referred to above. Briefly, the eye tracking system 21 includes an infra-red sensor assembly 20 and signal processing circuitry 22. The infra-red sensor assembly 20 is mounted on the headset 5 adjacent to the eye of the subject 7, as schematically indicated in FIGS. 1 and 2. FIG. 4 shows the details of the sensor assembly 20 in more detail. It will be seen that it includes an infra-red LED 16 mounted above the eye 23 of the subject 7 and a photo-transistor 17 which is sensitive to infra-red located beneath the eye 23. The LED 16 directs an infra-red beam at the lateral edge of the cornea 19 and sclera 18 border, the photo-transistor also being arranged to detect reflected infra-red light from this area. The photo-transistor 17 is coupled to provide input signals to the signal processing circuitry 22 which functions as an interface for the computer 1.

The gaze position as a function of time is calculated from the head position information supplied by the TrackIR system 12 and the eye tracking system 21. Gaze position measurements are calibrated for each subject 7 prior to the evaluation of a visual display. This is done by displaying a small target on the screen 3, such as a cross or a small circle at five locations in succession. These are the centre of the screen and the four diagonals of the screen, i.e. top left, top right, bottom left and bottom right. In each case, the target is located for 1 to 5 seconds in each location, preferably 1 second. This sequence is repeated twice. In the first instance, subjects are instructed to initially look directly ahead and not move their head as they follow the target with their eyes. During the second sequence, subjects are asked to follow the target by moving their head and not moving their eyes.

From these two sets of measurements, it is a straight forward task to calculate gaze location from the outputs of the head position and oculography systems.

The gaze position is determined by summing the relevant spherical polar coordinates available from the head position and oculography system 21. This is given by the following equations:

Θ_{gaze}=Θ_{head position}+Θ_{oculography} Equation 5

Φ_{gaze}=Φ_{head position}+Φ_{oculography} Equation 6

In the preferred system 50, the LED arrays 9 are controlled so that the light intensity therefrom varies sinusoidally under the control of control circuitry 6. The control circuitry 6 includes a waveform generator for generating the sinusoidal signal. In the event that the SSVER is used, the light from the LED array is conveyed to the visor via a fibre optic system. The circuitry 6 also includes amplifiers, filters, analogue to digital converters and a USB interface or a TCP interface or other digital interface for coupling the various electrode signals into the computer 1.

A translucent screen 10 is located in front of each LED array 9. Printed on the screen is an opaque pattern. The opacity is a maximum in a circular area in the centre of the centre of the screen. Beyond the circular area, the opacity falls off smoothly with radial distance from the circular area circumference, preferably, the opacity should fall off as a Gaussian function described by Equation 7. The screen reduces the flicker in the central visual field thus giving subjects a clear view of the visually presented material. The size of the central opaque circle should be such as to occlude the visual flicker in the central visual field between 1-4 degrees vertically and horizontally.

If r<R then P=1

If r≧R then P is given by the Equation 7 below.

$$P = {e}^{- {( r - R )}^{2} / {G}^{2}} \qquad Equation 7$$

where P is the opacity of the pattern on the translucent screen. An opacity of P=1.0 corresponds to no light being transmitted through the screen while an opacity of P=0 corresponds to complete transparency.

R is the radius of the central opaque disk while r is the radial distance from the centre of the opaque disk. G is a parameter that determines the rate of fall-off of opacity with radial distance. Typically G has values between R/4 and 2R. FIG. 5 illustrates the fall-off of opacity with radial distance from the centre of the disk. In FIG. 5, R=1 and G=2R. While a Gaussian fall-off of opacity with radius is preferable, any function that is smooth and has a zero gradient at r=R and at r>3G will be suitable.

The computer 1 includes software which calculates SSVEP or SSVER amplitude and phase from each of the electrodes in the headset 5 or MEG sensors.

Details of the hardware and software required for generating SSVEP and SSVER are well known and need not be described in detail. In this respect reference is made to the aforementioned United States patent specifications which disclose details of the hardware and techniques for computation of SSVEP. Briefly, the subject 7 views the video screen 3 through the special visor 4 which delivers a continuous background flicker to the peripheral vision. The frequency of the background flicker is typically 13 Hz but may be selected to be between 3 Hz and 50 Hz. More than one flicker frequency can be presented simultaneously. The number of frequencies can vary between 1 and 5. Brain electrical activity will be recorded using specialized electronic hardware that filters and amplifies the signal, digitizes it in the circuitry 6 where it is then transferred to the computer 1 for storage and analysis.

When using the SSVEP, brain electrical activity is recorded using multiple electrodes in headset 5 or another commercially available multi-electrode system such as Electro-cap (ECI Inc., Eaton, Ohio USA). When using the SSVER, commercial MEG recording system such as the CTF MEG System manufactured by VSM MedTech Ltd may be used. The number of electrodes or magnetic recording sites is normally not less than 8 and normally not more than 128, typically 16 to 32.

Brain electrical activity at each of the electrodes is conducted to a signal conditioning system and control circuitry 6. The circuitry 6 includes multistage fixed gain amplification, band pass filtering and sample-and-hold circuitry for each channel. Amplified/filtered brain activity is digitized to 16-24 bit accuracy at a rate not less than 300 Hz and transferred to the computer 1 for storage on hard disk. The timing of each brain electrical sample together with the time of presentation of different components of the audio-visual material are also registered and stored to an accuracy 10 microseconds. The equivalent MEG recording system that is commercially available performs the same functions.

### SSVEP and SSVER Amplitude and Phase

The digitized brain electrical activity (electroencephalogram or EEG) brain magnetic activity (MEG) together with timing of the stimulus zero crossings enables one to calculate the SSVEP or SSVER elicited by the flicker at a particular stimulus frequency from the recorded EEG or MEG or from EEG or MEG data that has been pre-processed using Independent Components Analysis (ICA) to remove artefacts and increase the signal to noise ratio. [Bell A. J. and Sejnowski T. J. 1995. An information maximisation approach to blind separation and blind deconvolution, Neural Computation, 7, 6, 1129-1159; T-P. Jung, S. Makeig, M. Westerfield, J. Townsend, E. Courchesne and T. J. Sejnowskik, Independent component analysis of single-trial event-related potential Human Brain Mapping, 14(3):168-85, 2001.]

Calculation of SSVEP or SSVER amplitude and phase for each stimulus cycle for a given stimulus frequency. Calculation accomplished used Fourier techniques using Equations 8 and 9 below.

$${a}_{n} = \frac{1}{S Δ τ} {\sum }_{i = 0}^{S - 1} f ( nT + i Δ τ ) cos ( \frac{2 π}{T} ( nT + i Δ τ ) ) \\ {b}_{n} = \frac{1}{S Δ τ} {\sum }_{i = 0}^{S - 1} f ( nT + i Δ τ ) sin ( \frac{2 π}{T} ( nT + i Δ τ ) ) \qquad Equation 8$$

Calculation of SSVEP Fourier components where aₙ and bₙ are the cosine and sine Fourier coefficients respectively. n represents the nth stimulus cycle, S is the number of samples per stimulus cycle (typically 16), Δτ is the time interval between samples, T is the period of one cycle and f(nT+iΔτ) is the EEG or MEG signal (raw or pre-processed using ICA).

$${SSVEP}_{amplitude} = \sqrt{( {A}_{n}^{2} + {B}_{n}^{2} )} or \\ {SSVER}_{amplitude} = \sqrt{( {A}_{n}^{2} + {B}_{n}^{2} )} \\ {SSVEP}_{phase} = a tan ( \frac{{B}_{n}}{{A}_{n}} ) or {SSVER}_{phase} = a tan ( \frac{{B}_{n}}{{A}_{n}} ) \qquad Equation 9$$

Where Aₙ and Bₙ are overlapping smoothed Fourier coefficients calculated by using Equation 10 below.

$${A}_{n} = {\sum }_{i = 1}^{i = N} {a}_{n + i} / N \\ {B}_{n} = {\sum }_{i = 1}^{i = N} {b}_{n + i} / N \qquad Equation 10$$

Amplitude and phase components can be calculated using either single cycle Fourier coefficients (aₙ and bₙ) or coefficients that have been calculated by smoothing across multiple cycles (Aₙ and Bₙ).

Equations 9 and 10 describe the procedure for calculating the smoothed SSVEP or SSVER coefficients for a single subject. For pooled data, the SSVEP or SSVER coefficients (Aₙ and Bₙ) for a given electrode are averaged (or pooled) across all of the subjects or a selected group of subjects.

As the number of cycles used in the smoothing increases, the signal to noise ratio increases while the temporal resolution decreases. The number of cycles used in the smoothing is typically in excess of 5 and less than 130.

The above equations apply to scalp SSVEP data as well as brain electrical activity inferred at the cortical surface adjacent to the skull and deeper regions. Activity in deeper regions of the brain such as the orbito-frontal cortex or ventro-medial cortex can be determined using a number of available inverse mapping techniques such as EMSE (Source Signal Imaging, Inc, 2323 Broadway, Suite 102, San Diego, Calif. 92102, USA) and LORETA (Pascual-Marqui R, Michel C, Lehmann D (1994): Low Resolution Electromagnetic Tomography: A New Method for Localizing Electrical Activity in the Brain Int J Psychophysiol 18:49-65). If the SSVER amplitude or phase changes at the specific brain regions listed above are used as the indicator of brain activity, then the multi-detector MEG recording system must be used in conjunction with an MEG inverse mapping technique (see Uutela K, Ha\ddot{}ma\ddot{}la\ddot{}inen M, Somersalo E (1999): Visualization of Magnetoencephalographic Data Using Minimum Current Estimates. Neuroimage 10:173-180 and Fuchs M, Wagner M, Kohler T, Wischmann H A (1999): Linear and Nonlinear Current Density Reconstructions, J Clin Neurophysiol 16:267-295).

While one or more subjects are viewing the images to be evaluated, the visual flicker is switched on in the visor 4 and brain electrical activity is recorded continuously on the computer 1.

FIG. 6 is a simplied flowchart showing a typical sequence of steps used in the method of the invention. The flowchart includes an initial step 70 in which the customer selects a visual display which is to be evaluated by the method of the invention. After the initial step, step 72 indicates the selection by the customer of the particular visual features F₁, F₂ . . . Fₙ of the visual display which are to be evaluated. The method then moves to step 74 in which the boundaries of the visual features F₁, F₂ . . . Fₙ are determined and these are then preferably expressed in terms of spherical polar coordinates, the datum of which is the centre of the screen 3. The method then moves to first question box 76 which determines whether the gaze of the subject, as determined by the head tracking system 12 and eye tracking system 21, is within the coordinate boundaries of visual feature F₁. If not, the method turns to a second question box 78 which determines a similar question with respect to the boundaries of visual feature F₂ and so on until the final question box 80 determines whether the gaze is within the boundaries of visual feature Fₙ. If no, then the sequence returns to the first question box 76 as shown.

If the gaze is within the boundary of visual feature F₁, then the software in the computer 1 determines the difference in brain activity from the reference level as indicated by step 82. The result is then accumulated in a running average step 88 and, at the end of the display sequence, step 94 indicates a graphical display of the average brain activity for visual feature F₁.

Similarly, where the gaze of the subject is determined to fall within the boundaries of the visual feature F₂, as determined by the second question box 78, the software determines the brain activity differences in step 84, the moving average in step 90 and generates the display in step 96. Similarly, if the gaze is determined to fall within the boundaries of visual feature Fₙ, as determined by question box 80, the software determines the difference in brain activity from the reference in step 86, the moving average in step 92 and generates the graphical display in step 98.

It will be appreciated that steps 82, 84 and 86 can be determined from different scalp sites so as to measure difference psychological responses, such as emotional responses, attention, long term memory encoding, engagement, desirability and likeability as described above. The various psychological responses are not separately shown for clarity of illustration. They can, however, be averaged and graphically displayed if required.

Further, the brain activity can be determined in various ways, as indicated above, including:

gamma or high frequency EEG or MEG activity;

frequency of EEG or MEG alpha activity; or

SSVEP or SSVER amplitude and phase measurements.

Where brain activity is determined by measuring SSVEP or SSVER, the amplitude and phase are preferably separately calculated for each subject at the end of the recording stage. Once all recordings are completed, group averaged data associated with specific gaze locations on the test object is calculated by averaging the smoothed SSVEP or SSVER amplitude and phase data from subjects to be included in the group (eg male, female, young, old) for different gaze locations on the test object. Separate group averages associated with predetermined gaze locations on the test object may then be calculated.

### EXAMPLE

Each subject 7 is seated before a video monitor and the headset 5 is placed on the subject's head. The visor 4 is then placed in position and adjusted so that the foveal block by the screens 10 prevents the appearance of the flicker over the screens 3 where the visual objects are presented. The head tracking system 12 and the eye tracking system 21 are then initialized, in accordance with the procedures described above. When pooling subjects to create the average response, the number of subjects whose data is to be included in the average should preferably be no less than 16.

Visual objects appear on the screen for different periods of time. Print and outdoor display material can be presented for 5 to 300 seconds depending on the amount of text while products and packaging can be presented as either a still image or rotating on a platform for 10 to 180 seconds. Architectural objects such as buildings, building interior and outdoor structures can be viewed as still images or animated sequences where the viewer moves through a path in space, similar to virtual reality.

In a typical study, one or more visual objects are presented to the subjects in a sequence. Each sequence of visual objects lasting no more than 300 seconds is followed immediately by a 30 second reference period in which a sequence of still images of scenery and a musical accompaniment. Typically, 60 images were presented over the period of 30 seconds with each image present for 0.5 seconds. The same sequence of images and music were presented after each sequence of visual objects. Brain activity levels during the adjacent scene images are used as a reference level for brain activity during the preceding visual objects. This enables removal of any long-term changes in brain activity that may occur over the time course of the recording period.

Pooled or averaged data at various brain sites associated with specific gaze locations on the test object can then be displayed to the client as the difference between the reference level and the value when participants are viewing specific locations on the visual object. A fixed offset between 0.2 to 0.6 preferably 0.3 radians is then added to the abovementioned difference to yield the SSVEP phase data at each scalp site.

FIG. 7 shows the visual object to be tested in accordance with the method of the invention. In this case, the visual object is a perfume bottle 100 having a main body 102, label 104, neck 106 and stopper 108. The purpose of the study was to determine the level of attractiveness of the bottle and to see what parts of the bottle are viewed more favourably than others. In this case, the perfume bottle 100 is selected to have two visual features for evaluation. The first visual feature is the upper part of the bottle which includes the neck 106 and stopper 108. The operator determines the boundary 110 of these visual features using standard software packages such as PowerPoint (Microsoft Corporation, One Microsoft Way, Redmond, Wash. 98052, USA) or CorelDraw (Corel Corporation, 1600 Carling Avenue, Ottawa, Ontario K1Z 8R7, Canada) and these are stored in the memory of the computer 1. A second part of the image of the object is then selected for evaluation. In this case it is the main body 102 of the bottle and the boundaries are determined, as indicated by boundary line 112. The coordinates of the boundary line 112 are entered in the memory of the computer 1, as before.

The display sequence is presented to the subjects 7 and the brain activities are measured and recorded, in accordance with the procedures described above and the results plotted, as described below.

FIG. 8 shows the brain activity for the upper part of the bottle which includes the neck 106 and stopper 108. It will be seen from FIG. 8 that there are relatively high levels of global attention (associated with aesthetic judgments), engagement and desirability.

FIG. 9 graphically illustrates activity associated with the main body 102 of the bottle. It will be seen that FIG. 9 shows that there is elevated levels of global attention and desirability.

In this example, the client would be advised that the design is attractive to the target audience and that the body of the bottle is especially attractive. Any changes to the specific design of this bottle should avoid those regions already considered attractive and desirable.

Many modifications will be apparent to those skilled in the art without departing from the spirit and scope of the invention.

1. A method of evaluating the response of a subject to visual features of a visual display, the method including the steps of:
(a) presenting a visual display having particular visual features to the subject during a first period;
(b) determining brain activity of the subject during the first period;
(c) presenting reference display material to a subject during a second period;
(d) determining reference brain activity of the subject during the second period;
(e) tracking the gaze position of at least one of the eyes of the subject on the visual display during the first period; and
(f) evaluating the response of the subject to particular visual features of the visual display by determining differences in brain activity determined between steps (b) and (d) when the gaze of the subject is directed at the particular features.

2. A method as claimed in claim 1 wherein the visual display is printed advertising material, text layout, product design, packaging website, the interior or exterior of a building.

3. A method as claimed in claim 2 wherein the visual display is displayed on a video screen.

4. A method as claimed in claim 3 including the step of selecting the visual features of the visual display and determining the areas where the selected visual features are located on the video screen and wherein step (e) determines when the gaze position of the subject falls upon respective area of the selected visual features on the video screen.

5. A method as claimed in claim 4 wherein the differences in brain activity determined in step (f) are averaged for each selected visual feature.

6. A method as claimed in claim 1 wherein steps (a) to (e) are presented to a plurality of subjects and step (f) includes the steps of averaging the differences in brain activities of the subjects.

7. A method as claimed in claim 1 wherein steps (b) and (d) are carried out by determining gamma or high frequency EEG or MEG activity.

8. A method as claimed in claim 1 wherein steps (b) and (d) are carried out by detecting EEG or MEG activity in the frequency range 8 to 13 Hz.

9. A method as claimed in claim 1 wherein steps (b) and (d) are carried out by assessment of the phase of steady state visually evoked potentials (SSVEP) in EEG signals obtained from the subject or subjects or by assessment of steady state visually evoked responses (SSVER) in MEG signals obtained from the subject or subjects.

10. A method as claimed in claim 1 wherein steps (a) and (c) include the steps of placing electrodes at scalp sites to obtain output EEG signals which enable assessment of:
visual attention to detail of the visual features;
emotional intensity associated with the visual features;
long term memory encoding associated with the visual features;
engagement with the visual features;
attraction associated with the visual features;
desirability associated with the visual features; and/or
likeability associated with the visual features.

11. A method as claimed in claim 10 including the step of applying a sinusoidally varying visual flicker stimulus to each subject during steps (a) and (c) to thereby enable calculation of Fourier coefficients from said output signals to thereby enable calculation of said SSVEP amplitudes and/or phase differences.

12. A method as claimed in claim 11 wherein said SSVEP amplitude and phase are calculated by the equations:
$${SSVEP}_{amplitude} = \sqrt{( {A}_{n}^{2} + {B}_{n}^{2} )}$$
$${SSVEP}_{phase} = a tan ( \frac{{B}_{n}}{{A}_{n}} )$$
where: aₙ and bₙ are cosine and sine Fourier coefficients calculated by the equations:
$${a}_{n} = \frac{1}{S Δ τ} {\sum }_{i = 0}^{S - 1} f ( nT + i Δ τ ) cos ( \frac{2 π}{T} ( nT + i Δ τ ) )$$
$${b}_{n} = \frac{1}{S Δ τ} {\sum }_{i = 0}^{S - 1} f ( nT + i Δ τ ) sin ( \frac{2 π}{T} ( nT + i Δ τ ) )$$
where:
aₙ and bₙ are the cosine and sine Fourier coefficients respectively where;
n represents the nth flicker stimulus cycle;
S is the number of samples per flicker stimulus cycle;
Δτ is the time interval between samples;
T is the period of one cycle;
f(nT+iΔτ) is the EEG signal (raw or pre-processed using ICA) obtained from said predetermined scalp sites;
and wherein Aₙ and Bₙ are overlapping smoothed Fourier coefficients calculated by using the equation:
$${A}_{n} = {\sum }_{i = 1}^{i = N} {a}_{n + i} / N$$
$${B}_{n} = {\sum }_{i = 1}^{i = N} {b}_{n + i} / N$$

13. A method as claimed in claim 12 including the steps of:
obtaining EEG signals from a plurality of scalp sites of each subject; and
utilising inverse mapping techniques such as BESA, EMSA or LORETA to produce modified EEG signals which represent activity in deeper regions of the brain of each subject such as the orbito-frontal cortex or the ventro-medial cortex.

14. A method as claimed in claim 12 including the step of averaging the Fourier coefficients Aₙ and Bₙ for a selected group of subjects and then calculating the SSVEP amplitudes and SSVEP phase differences for said group of subjects.

15. A method as claimed in claim 11 wherein the flicker signal is applied only to the peripheral vision of each subject.

16. A method as claimed in claim 15 including the steps of directing the flicker signal towards the eyes of each subject via first and second screens and wherein each screen includes an opaque area, and wherein the method further includes the step of positioning the screens to the relative position of each subject such that said opaque areas prevent said flicker signal impinging on the fovea of each eye of each subject.

17. A method as claimed in claim 16 wherein the opacity of each screen decreases as a function of distance from its opaque area so that the intensity of the flicker signal impinging on each retina of each subject decreases in value from the central vision to the peripheral vision.

18. A method as claimed in claim 17 including the step of applying a masking pattern to each screen to define the opacity thereof, the method including the step of applying the pattern in accordance with a masking pattern function which provides zero or low gradients for changes in opacity adjacent to its opaque area and peripheral areas thereof which define parts of the flicker signal impinging on the peripheral vision of each subject.

19. A method as claimed in claim 18 wherein the opaque area of each screen is circular and wherein the masking pattern function is selected to be a Gaussian function, so that the opacity P of the screen is defined by the equation:
$$P = {e}^{- {( r - R )}^{2} / {G}^{2}}$$
where:
r is the radial distance from the centre of the opaque area; and
G is a parameter that determines the rate of fall-off of opacity with radial distance, and wherein when r<R, P=1.

20. A method as claimed in claim 19 wherein G has a value in the range R/4 and 2R.

21. A method as claimed in claim 12 including the steps of applying an electrode to the scalp of each subject at the O₁ site, calculating SSVEP amplitudes and phase differences from EEG signals from said electrode whereby the output signals indicate each subject's visual attention to details of the selected visual features.

22. A method as claimed in claim 13 including the step of utilising inverse mapping determines brain activity in the left cerebral cortex in the vicinity of Brodman's area 17 whereby the modified output signals indicate each subject's visual attention to details of the selected visual features.

23. A method as claimed in claim 12 including the step of applying an electrode to the scalp of each subject at a site which is approximately equidistant from sites O₂, P₄ and T₆, calculating SSVEP amplitudes and phase differences from EEG signals from said electrode whereby the output signals indicate each subject's emotional intensity associated with the selected visual features.

24. A method as claimed in claim 13 wherein the step of utilising inverse mapping determines brain activity in the right cerebral cortex in the vicinity of the right parieto-temporal junction whereby the output signals indicate each subject's emotional intensity associated with the selected visual features.

25. A method as claimed in claim 12 including the steps of applying an electrode to the scalp of each subject at the F₃, F₄, Fₚ₁ and Fₚ₂ sites, calculating SSVEP amplitudes and phase differences from EEG signals from said electrodes, calculating values for attraction-repulsion using the equation:
attraction=(a₁*SSVEP phase advance at electrode F₃+a₂*SSVEP phase advance at electrode Fₚ₁−a₃*SSVEP phase advance at electrode F₄−a₄*SSVEP phase advance at electrode Fₚ₂)
where a₁=a₂=a₃=a₄=1.0
whereby said values indicate each subject's attraction or repulsion towards the selected visual features.

26. A method as claimed in claim 13 wherein the step of utilising inverse mapping determines brain activity in:
the right orbito-frontal cortex in the vicinity of Brodman area 11;
the right dorso-lateral prefrontal cortex in the vicinity of Brodman area 9;
the left orbito frontal cortex in the vicinity of Brodman area 11; and
the left dorso-lateral prefrontal cortex in the vicinity of Brodman area 9; and
calculating a value for attraction-repulsion using the equation:
attraction=(c₁*right orbito-frontal cortex (in vicinity of Brodman area 11)+c₂*right dorso-lateral prefrontal cortex (in vicinity of Brodman area 9)+c₃*left orbito frontal cortex (in vicinity of Brodman area 11)+c₄*left dorso-lateral prefrontal cortex (vicinity of Brodman area 9))
where c₁=1, c₂=1, c₃=1, c₄=1,
whereby said values indicate each subject's attraction or repulsion towards the selected visual features.

27. A method as claimed in claim 12 including the steps of applying electrodes to the scalp of each subject at F₃, F₄, Pₚ₁ and Fₚ₂ sites, calculating SSVEP amplitudes and phase differences from said electrodes, calculating values for engagement in features of the advertisement by a weighted mean SSVEP phase advance at said sites using the equation:
engagement=(b₁*SSVEP phase advance at electrode F₃+b₂*SSVEP phase advance at electrode Pₚ₁+b₃*SSVEP phase advance at electrode F₄+b₄*SSVEP phase advance at Electrode Fₚ₂)
where b₁=0.1, b₂=0.4, b₃=0.1, b₄=0.4,
whereby said values indicate each subject's engagement in the selected visual features.

28. A method as claimed in claim 13 wherein the step of utilising inverse mapping determines brain activity in:
the right orbito frontal cortex in the vicinity of Brodman area 11;
the right dorso-lateral prefrontal cortex in the vicinity of Brodman area 9;
the left frontal cortex in the vicinity of Brodman area 11; and
the left dorso-lateral prefrontal cortex in the vicinity of Brodman area 9,
calculating SSVEP amplitudes and phase differences from said modified EEG signals from said electrodes; and
calculating a value for engagement using the equation:
engagement=(d₁*right orbito frontal cortex (in vicinity of Brodman area 11)+d₂*right dorso-lateral prefrontal cortex (in vicinity of Brodman area 9)+d₃*left orbito frontal cortex (in vicinity of Brodman area 11)+d₄*left dorso-lateral prefrontal cortex (in vicinity of Brodman area 9))
where d₁=0.1, d₂=0.4, d₃=0.1, d₄=0.4,
whereby said values indicate each subject's engagement in the selected visual features.

29. A method as claimed in claim 3 wherein step (e) includes:
fitting a headset to the subject or subjects having electrodes therein for determining the brain activities of steps (a) and (c);
tracking movements of an eye of each subject relative to his or her headset to generate eye position signals;
tracking the movements of the head of each subject relative to the video screen to produce head position signals; and
combining said eye position and head position signals to thereby determine the gaze position of each subject relative to a reference point on the video screen.

30. A system for evaluating the response of a subject to visual features of a visual display, the system including:
(a) display means for displaying said visual features to the subject;
(b) means for determining brain activity of the subject at predetermined scalp sites of the subject;
(c) gaze tracking means for determining the gaze position of the subject on said display means;
(d) detecting means for detecting when the gaze position of the subject impinges on selected visual features; and
(e) averaging means for calculating average values of brain activity for each of the selected visual features when the detecting means detects that the gaze position of the subject impinges on the respective selected visual features.
